(19) United States
(12) Patent Application Publication
(10) Pub. No.: US 2024/0269393 A1
(43) Pub. Date: Aug. 15, 2024

(54) Systems, Methods and Devices for Portable Medication Delivery

(71) Applicant: Eric T. Strand, Boston, MA (US)

(72) Inventor: Eric T. Strand, Boston, MA (US)

(21) Appl. No.: 18/439,746

(22) Filed: Feb. 12, 2024

Related U.S. Application Data
(60) Provisional application No. 63/484,539, filed on Feb. 13, 2023.

(51) Int. Cl. A61M 5/32; A61M 5/20

(52) U.S. Cl. CPC A61M 5/3243; A61M 5/20

(57) ABSTRACT

Medication delivery devices, systems, and methods that use an autoinjector, having an armed injection mechanism operable to inject a needle into a patient intramuscularly or subcutaneously and dispense a predetermined dose of medication or treatment fluid. In some embodiments, a safety locking mechanism may be employed to prevent accidental actuation, and once armed, a trigger mechanism is operable through contact with the patient's body to activate the device and dispense medication or treatment fluid. A needle cover may be deployed after usage to prevent reuse or accidental needle sticks. An exemplary device may be compliant with ISO/IEC 7810/ID-1/CD80 international credit card size standardizations of 85.60 mm length by 53.98 mm width, with a thickness of less than or equal to 3.80 mm (5 credit cards thick) to enable discrete, easy transport in virtually all commercially available wallets and cardholders with no further modifications or compromises required.

### CROSS REFERENCE TO RELATED APPLICATIONS

Priority is claimed to U.S. Provisional Patent Appl. No. 63/484,539, filed Feb. 13, 2023, which is incorporated by reference herein in its entirety.

### FIELD OF THE INVENTIONS

The inventions described herein generally pertain to the field of medication or treatment fluid delivery, and to devices for inserting medicines into the body, and to the manner of making and using the same or components thereof. More particularly, illustrative embodiments described herein relate to devices for delivery of medication or treatment fluid contained within a prefilled autoinjector, such as an autoinjector having a form factor or volumetric shape approximately the size of a credit card.

### INTRODUCTION

Exposure to allergens, toxins, and other substances can have serious consequences for some individuals and animals. For instance, some people are highly allergic to peanuts, shellfish, specific drugs, and bee venom, among others, leading to anaphylactic shock and other symptoms. To avoid injury or death, a prompt response is necessary. For example, administering epinephrine can relieve the symptoms of anaphylaxis, while injecting an antidote can reduce the harm of a nerve agent exposure. Similarly, rapid injection of drugs like beta blockers, blood thinners, and antihistamines can provide relief from various medical conditions. It is necessary for members of the public with such allergies or medical conditions to carry their medication with them at all times. Commercially available autoinjectors are generally bulky and difficult to carry in a discrete manner, which may lead to members of the public forgetting to or foregoing carrying their medication with them, and subsequent substantial injury or death. Therefore, a thin, sleek, inexpensive, and reliable autoinjector device able to be discreetly stored is needed to enhance public safety and prevent unneeded harm.

So as to reduce the complexity and length of this specification, the materials identified in the following paragraphs in this section are herein expressly incorporated by reference in their entirety. The incorporated material is believed to be non-essential in accordance with 37 CFR 1.57 because it is referred to for purposes of providing general support, background, or information relating to the inventions. However, if any such material is deemed essential under Rule 1.57, any such text will be expressly added herein pursuant by the applicable rules.

U.S. Pat. No. 9,138,539 may generally concern an injector device for the percutaneous injection of fluids.

U.S. Pat. No. 7,390,319 may generally concern an automatic needle injector having a safety triggering mechanism.

U.S. Pat. No. 10,398,842 may generally concern a rectangular shaped auto-injection syringe.

U.S. Pub. No. 2018/0008774 may generally concern devices, systems and methods for medicament delivery.

U.S. Pub. No. 2019/0175837 may generally concern medicament delivery devices for administration of a medicament within a prefilled syringe.

U.S. Pub. No. 2019/0307955 may generally concern fluid delivery device needle retraction mechanisms, cartridges and expandable hydraulic fluid seals.

Int. Pub. No. 2016/160341 may generally concern a compartmentalized auto-injector system.

JP Pat. No. 7366974 may generally concern an auto-injector for subcutaneous administration of drugs.

The document titled AdrenaCard, an Epinephrine Autoinjector The Size of a Credit Card may generally concern an autoinjector similar in size to a credit card. (AdrenaCard, an Epinephrine Autoinjector The Size of a Credit Card (2016), available at the legacymedsearch homepage).

The document titled “Adrenaline auto-injectors for the treatment of anaphylaxis with and without cardiovascular collapse in the community may generally concern potential problems with adrenaline auto-injector use.” (A. Sheikh et al., Adrenaline auto-injectors for the treatment of anaphylaxis with and without cardiovascular collapse in the community (2012), CD008935).

The document titled “Consider AUVI-Q, the Pocket-Sized Epinephrine Auto-Injector Made to be Carried (Sponsored) may generally concern epinephrine injectors that are easy to use and carry.” (Kaleo, Consider AUVI-Q, the Pocket-Sized Epinephrine Auto-Injector Made to be Carried (Sponsored) (2022), available on the snacksafely website).

Unless specifically noted, words and phrases in this specification and the claims are to be given their relevant plain and ordinary English meaning. Thus, except where this specification uses the exact phrase “[word or phrase] is hereby defined to mean [definition],” the inventor expressly elects, as lexicographer, to use the plain and ordinary meaning of words in the specification and claims rather than special definitions. Absent such specific statement to apply a special definition, the plain and ordinary meaning applies to the terms used in the specification and claims.

The inventor is also aware of the normal precepts of English grammar. Thus, if a noun, term, or phrase is intended to be characterized, specified, limited, broadened, modified or narrowed in some way, then such noun, term, or phrase will expressly include any desired or intended adjectives, descriptive terms, or other modifiers in accordance with the normal precepts of English grammar. Absent the use of such adjectives, descriptive terms, or modifiers in the claim language, it is intended that such nouns, terms, or phrases be given their ordinary and customary meaning as set forth above.

Further, the inventor is aware of the availability and limits of functional claiming under 35 U.S.C. § 112(f). As used herein or in the claims, the words “function,” “means” or “step” do not indicate an intent to invoke the special provisions 35 U.S.C. § 112(f) to define the inventions. To the contrary, if the provisions of 35 U.S.C. § 112(f) are sought to be invoked to define the inventions, the claims will specifically and expressly recite the exact phrase “means for” and will also expressly recite the word “function” followed by a description of the function (i.e., will state: “means for performing the function of [insert function]”), without also reciting in such phrases any structure, material or act in support of the function. Thus, even if a claim recites a “means for performing the function of . . . ” if a claim also recites any supporting structure, material or acts then it is the clear intention of the inventor not to invoke the provisions of 35 U.S.C. § 112(f). If the provisions of 35 U.S.C. § 112(f) are invoked to define the claimed inventions (using the technique defined above), it is intended that the inventions not be limited only to the specific structure, material or acts that are described in any specific embodiment, but in addition, include any equivalent structures, materials, or acts that perform the claimed function, or any structures, materials, or acts described in any alternative situations or forms of the inventions, or that are within the appropriate limits of claim scope and construction and that are reasonably described and reasonably enabled by this specification.

The inventions described in this specification and recited in the claims are not directed to laws of nature, natural phenomena, or abstract ideas, but instead, are directed to one or more of the expressly permitted statutory categories of inventions, i.e., processes, machines, manufactures, or compositions of matter. Nor are the inventions claimed herein directed to any prohibited examples of abstract ideas such as fundamental economic practices, methods of organizing human activity, an idea itself, or any mathematical relationships/formulas. The claimed inventions are directed to significantly more than any abstract idea by itself and include specifically claimed inventive concepts so as to not preempt any fundamental building blocks of human ingenuity.

Numerous possible or potential aspects, objects, modifications, features, uses, or advantages of various inventions described herein will be apparent to those of ordinary skill in the art from this specification, drawings, and claims. However, without characterizing or limiting the scope of the various inventions as they are claimed, some of the possible or potential aspects, objects, features, uses, or advantages of various inventions are summarized below. None of the following possible or potential aspects, objects, features, uses, or advantages are a disavowal, disclaimer, characterization, or interpretation of any aspects of any of the claims. These possible or potential aspects, objects, features, uses, or advantages might apply to any or none of the claimed inventions.

It is or may be an object, goal or advantage of some of the inventions to enable for discreet carrying of medication injectors.

It is or may be an object, goal or advantage of some of the inventions to enable medication injectors to fit inside all commercially available wallets and cardholders with no modifications required.

It is or may be an object, goal or advantage of some of the inventions to increase public safety, medicine efficacy, carrying convenience, among other uses and benefits.

### BRIEF DESCRIPTION OF THE DRAWINGS

Additional understanding of the inventions may be derived by referring to the description when considered in connection with the figures. In the figures, like reference numbers refer to like elements or acts. While the figures provide several examples of aspects, elements, modifications, or components that may or may not be variously involved with the systems, methods, and devices described herein, the figures are not provided to define, limit, or affect the scope of the inventions claimed or described herein. Rather, the words of the claims themselves are intended to define and control the scope of the inventions claimed herein. The figures are to assist the reader in understanding aspects of the various inventions disclosed and claimed herein.

FIG. 1 shows a partially cross-sectional schematic representation of elements of an example injecting device structured according to certain principles of the invention, in an assembled and stowed state, shown with the body lid moved superior.

FIG. 2 shows a view of the example of FIG. 1, in an assembled state, with additional depictions of internal and obscured elements.

FIG. 3 shows a proximal end view of the example of FIG. 2.

FIG. 4 shows a perspective view of an assembled injection device.

FIG. 5 shows a schematic representation of mechanistic elements of the injection device in FIG. 1, with autoinjector body and springs omitted for improved understanding.

FIG. 6 shows a perspective view of the assembled injection device of FIG. 1, in a set-up position with safety lock removed.

FIG. 7 shows a perspective view of the assembled injection device of FIG. 1, in a fully activated and dispensing position with safety lock removed and release mechanism actuated.

FIG. 8 shows a perspective view of a release mechanism such as that illustrated in FIG. 1, but in a further state of assembly for clarity.

FIG. 9 shows a perspective view of a needle insertion mechanism such as that illustrated in FIG. 1, but in a further state of assembly for clarity.

FIG. 10 shows a perspective view of a medication dispensing vessel powered by gas, spring bias, or other activation mechanisms, such as that illustrated in FIG. 1, but in a further state of assembly for improved understanding.

FIG. 11 shows a flowchart of an example of a method of use of an injection device.

FIG. 12 shows a needle cover within the outer proximal edge of an assembled injection device body.

FIG. 13 shows a needle cover slid over a deployed needle after an injection device is actuated.

Elements and acts in the figures are illustrated for simplicity, may not be to scale, and have not been rendered according to any particular embodiment or example and are not to depict any essential or required limitations.

### ADDITIONAL DETAILED DESCRIPTION

The inventions are explained in additional detail below.

Due to the unpredictable nature of medical emergencies, including conditions such as but not limited to anaphylaxis, hypoglycemia, envenomation, or chemical intoxication, it is imperative that at-risk patients carry with them lifesaving medication at all times.

However, medications and dispensing devices often fail to fit into most commercially available wallets or credit card holders, or may otherwise require unique external housings, custom carrying cases, or adaptors that may be cumbersome to the user. For example, cylindrical medication vessels have fundamental diameters and volume relationships and, therefore, limit medication doses able to be delivered if scaled down to sizes approximating the dimensions of a credit card thin enough to fit into a commercially available wallet without the need for external carrying cases or adaptors. Indeed, various structural designs may partially or entirely fail if scaled down in size.

With regard solely to anaphylaxis, one of the most common reasons for a patient to carry a portable medication autoinjector, recent research has shown that 52% of patients reported not having an autoinjector with them during their most severe allergic reaction. (Ann Allergy Asthma Immunol. 2018; 121(4):479-489). Less than half of patients reported consistent quick (<5 min) access to an autoinjector, and 38% of patients cited difficulty in remembering to carry their autoinjector.

These data taken in concert with the inherent designs of prior devices represent a failure of others to solve a long-felt medical need within our society.

To design a device capable of delivering variable doses of medication with a modifiable injection depth contained within a single housing or body substantially approximating the dimensions of a credit card with a thickness suitable for realistic transportation of said device in commercially available wallets or credit card holders without the need for external carrying cases, housings, or adaptors, several major and unexpected barriers had to be overcome.

For example, to serve its primary function, I designed devices to approximate the current dimensions of a credit card and be thin enough to realistically fit in commercially available credit card holders without modification or compromise. As the average consumer may generally carry several cards at a time on average, a thickness of five credit cards (3.8 mm) was felt to be of reasonable thickness, though this statement is not meant to be limiting.

At such dimensions, material science limitations are imperative to consider no matter which manufacturing material is ultimately chosen. A device substantially two-dimensional in nature had particularly complex design challenges to overcome in order to meet the demands of a user while retaining long-term functionality. For example, the design of the housing alone required extensive calculations to meet structural requirements to withstand the expected forces of users who may, for example, keep said device within a wallet in a back pocket for months or years. Further, to ensure the housing would not warp and inhibit device functionality over long periods of time, customized material science topology degradation software was employed.

The described near completely two-dimensional medication vessel kept under pressure required complex proprietary wall strength calculations and design optimizations using original software to ensure, among other things, the pressures required for proper function would be withstood at sub-millimeter scale. The medication vessel was designed, in certain instances, such that a variety of medication dosages and volumes are able to be delivered without modification to the device.

As no prior available microfluidic tubing fit the needs of the device, a custom small diameter microfluidic tubing was manufactured and tested. Further, custom small diameter springs needed to be wound in accordance with force calculations taking into account strength limits of surrounding materials as well as the standard of care for medication administration into a patient.

A custom needle with proper length, diameter, and flow rates given the medication volume needed to be fashioned. The length of the custom needle was tailored to administer a variety of intramuscular or subcutaneous injections, without need for extensive modifications to the device.

A pressure resistant seal that could be easily punctured to allow for the flow of medication yet withstand the significant pressures required over time and without failure needed to be fashioned as no commercially available seal existed.

Each part had to be designed within strict dimensional parameters, and every part was subject to rigorous computational analysis to ensure optimal function at sub-millimeter scales. In some examples, parts were uniquely design to avoid extending beyond an external housing, and all or substantially all available area within the housing was considered, used or configured with careful consideration of the structural requirements of the device.

Put together, these advancements, designs, and features create self-contained robust devices with many possible medications and dosage variations in a realistically portable footprint able to fit within commercially available credit card holders and wallets without modification or compromise. The inventions support patient access to life-saving medication at all times for unexpected events, thereby serving to address a unique but fundamental medical necessity not able to be solved by others.

For the purposes of explanation, “proximal” refers to an active end of the autoinjector through with the needle and release mechanisms arise and at which a patient's body interfaces with the autoinjector, while “distal” refers to an opposite edge at which is furthest from a patient's body at the time of activation.

The inventions may be structured to comply with the exact dimensions of the ISO/IEC 7810/ID-1/CD80 international credit card size standardizations of 85.60 mm length by 53.98 mm width and a thickness of less than or equal to 3.80 mm (5 credit cards thick) to enable easy transport in commercially available wallets and cardholders with no modifications required. Assembly may include an autoinjector body which houses the dynamic components of an autoinjector mechanism, and a multi-component mechanism which transfers force from contact with the patient's body to initiate the dispensing of a dose of medication or treatment fluid through a needle intramuscularly or subcutaneously into a patient's body.

An autoinjector can include a needle-insertion mechanism structured to drive a needle into the patient's body by a fixed distance and at a desired injection site; a safety locking mechanism configured to prevent undesired actuation of the needle-insertion mechanism; a medication dispensing vessel associated with the needle-insertion mechanism and operable to dispense the dose of fluid from a quantity of fluid confined inside the vessel; a microfluidic tubing or channel tract coupled to the medication dispensing vessel and the needle-insertion mechanism; and a trigger mechanism operable to actuate the needle-insertion mechanism and initiate medication flow from the medication dispensing vessel.

A needle-insertion mechanism can be structured to automatically drive the needle into a patient's body responsive to actuation of the trigger. A needle-insertion mechanism may include injection springs compressed upon assembly of the apparatus in a way that provides the necessary force needed to drive the needle in the dispensing direction upon actuation. Alternatively, an injection spring may employ one or more compliant mechanisms, or flexures.

An actuation mechanism may be structured for actuation by way of the patient urging the autoinjector in a direction substantially perpendicular to the surface of a human body at the injection site. One such actuation mechanism includes a winged latch that is activated upon removal of a safety lock mechanism, which when activated displaces a component supporting a compressed trigger holding a main needle-insertion mechanism in tension by maintaining compression of an injection spring.

A trigger mechanism may further enable a release mechanism structured to displace a latch with respect to a trigger to permit relative motion of the needle-insertion mechanism with respect to a housing. A release mechanism may include a component body enabling two articulating wings to fit into a secondary release component supporting a main trigger mechanism. Upon safety lock removal, a main release body is forced proximally, extending outside of an autoinjector body due to the movement of compressed springs. In the same motion, winged latches may be withdrawn from a secondary release mechanism component, which enables pressure from subsequent patient activation to translate through the displaced wings and further displace a secondary release mechanism from a main trigger. A main trigger is then freely displaced due to the action of compressed springs, which enables a main needle-insertion mechanism to activate and slide forward, driving two needle-insertion components together and forward out a proximal patient facing side of an autoinjector and initiating the flow of medication from a medication dispensing vessel.

In some situations, device assembly can include a safety locking mechanism structured to avoid undesired actuation of both a release mechanism and a needle-insertion mechanism. One safety locking mechanism includes a textured strip that can be removed from a blocking position at which the strip contains a release mechanism in its compressed state with latch wings stowed, and additionally blocks a proximal component of a needle-insertion mechanism. In some embodiments, an autoinjector assembly may include a locking mechanism structured to resist re-use of a device or accidental post-usage needle sticks. One locking mechanism may be structured to carry a needle cover within the outer edge of the housing and permit a user to move the housing cover into a protective position covering the needle after use of an autoinjector. The locking mechanism may be automatically actuated when the device assembly is first used to inject a dose of treatment fluid. Certain autoinjector assemblies may include a seal over the opening through which a needle will pass when activated in order to cover and maintain sterility of a needle before use. In some implementations, a sheath is included and is structured to permit penetration by the needle subsequent to actuation of the trigger.

Certain examples of the inventions may include a needle-insertion mechanism that is operable to drive a needle into a patient upon actuation of a release mechanism and subsequent trigger mechanism, while also initiating the flow of medication from a medication dispensing vessel. According to some aspects, a needle-insertion mechanism may be structured for automatic operation and dispensing of a dose of medication subsequent to actuation. A needle-insertion mechanism may be structured to contain a two-component system. The proximal component may contain a needle to be driven into the patient with a depression for safety strip insertion to prevent premature needle release, and a sharp yet hollow cone on the distal side facing the distal component to enable seal puncture and medication release from a medication dispensing vessel upon needle-insertion mechanism activation from trigger actuation. There may be a thin seal proximal to a needle tip to ensure sterility before injection. A distal component may contain a depression enabling a trigger assembly to block its forward movement until activation, a distal facing face enabling compression springs or other forward-movement forcing mechanisms to interface, a proximal facing housing containing an o-ring and seal that upon needle-insertion mechanism release interface with a proximal component to puncture a seal and initiate medication flow from a medication dispensing vessel, and an interface immediately distal to this seal to enable a microfluidic tubing or channel tract from a medication dispensing vessel to translate a the needle-insertion mechanism and interface with a seal.

A medication dispensing vessel may be structured to contain a two part enclosure enabling a largely rectangular barrier to fit inside and move freely in one dimension. A barrier may be structured to contain two recessions enabling a lubricating compound to be applied and aid in free movement. A top vessel component may be structured to contain an opening that interfaces with microfluidic tubing or channel tract to enable medication flow from a dispensing vessel powered by gas, spring bias, or other activation mechanisms to a needle-insertion mechanism and out through a needle into a patient. Gas (such as carbon dioxide, propane, or butane, although many others can be used) may be placed in a condensed form within the bottom half of the dispensing vessel, with a barrier fit into the vessel, and a top vessel component slid onto a bottom vessel component creating a seal with complementary interfacing structure connection. One or more compressed springs may also or instead be used to place the barrier in tension. Medication may then be loaded into the top of a completed dispensing vessel and sealed with a microfluidic tubing or channel tract. As a microfluidic tubing or channel tract interfaces with the distal component of a needle-insertion mechanism at a puncturable seal, a dispensing vessel then forms a closed system which is pressurized due to the expansion of the gas, springs, or other activation mechanisms used to charge the vessel. Upon puncturing a needle-insertion seal due to actuation of a release mechanism and trigger mechanism, a closed system is broken, and a gas, springs, or other activation mechanism in the vessel is able to expand thereby pushing medication out from the top of a vessel and out through a tubing or channel tract and needle and into a patient.

An invention may be embodied as a device structured to provide auto-injection functionality when triggered by a user. An exemplary such device includes an auto injector body, a needle-insertion mechanism, a release mechanism, a trigger mechanism, a medication dispensing vessel with microfluidic tubing, and a safety locking mechanism. An autoinjector can be configured to dimensions that comply with ISO/IEC 7810/ID-1/CD80 international credit card size standardizations of 85.60 mm length by 53.98 mm width and a thickness of less than or equal to 3.80 mm (5 credit cards thick) to enable easy transport in all commercially available wallets and cardholders with no modifications required. A needle-insertion mechanism may be structured to drive a needle by a fixed distance into a patient and initiate the dispensing of a dose of treatment fluid or medication into a patient. A release mechanism can be operable by a patient to initiate the actuation of a trigger mechanism, which then initiates and enables operation of a needle-injection mechanism and a medication dispensing vessel. A locking safety strip mechanism may be structured to both prevent arming a release mechanism to prevent accidental trigger actuation and to provide steric blocking of the proximal component of a needle-insertion mechanism.

An invention may be embodied as a method for a patient to inject a dose of medication or treatment fluid. One such method includes providing an autoinjector structured with dimensions that comply with ISO/IEC 7810/ID-1/CD80 international credit card size standardizations of 85.60 mm length by 53.98 mm width and a thickness of less than or equal to 3.80 mm (5 credit cards thick) to enable easy transport and storage in all commercially available wallets and cardholders. Said autoinjector may include a dispensing end; operating removal of a safety locking strip permits a release mechanism of an autoinjector to become set up and extend away from the body of an autoinjector. Depressing a release mechanism leads to activation of a trigger mechanism which then permits actuation of a compressed or biased needle-insertion mechanism that drives a needle a fixed distance into a patient and initiates dispensing the dose of medication or treatment fluid intramuscularly or subcutaneously; orienting the autoinjector substantially perpendicular to the skin of a patient at a desired injection site; pressing a dispensing end against the skin to depress an armed release mechanism by placing force against an armed release mechanism, ideally through contact with the body near the intended site of injection; actuating an internal trigger mechanism and subsequent needle-insertion and medication injection via release mechanism depression; holding the autoinjector in its activated state on the body with a needle inserted to enable full medication dispense; removing the needle from the body and discarding an autoinjector in a properly suitable location.

A device for administering medication to a patient may comprise of a device body having exactly, substantially, or approximately the dimensions of a credit card, or approximately 85.60 mm in length, and a width of approximately 53.98 mm, and a thickness of approximately less than or equal to 3.80 mm. A device may include a safety locking mechanism within a device body that can employed to prevent accidental actuation of a needle, wherein a safety locking mechanism can include a safety locking strip positionable from a first armed position to a second disarmed position.

Once a safety locking mechanism is set up, a trigger mechanism may be operable through contact with a patient's body to actuate a device and dispense a predetermined dose of medication or treatment fluid to the patient. A trigger mechanism can comprise of a catching interface mechanism.

A device can comprise of a generally rectangular medication dispensing vessel that can be associated with a needle-insertion mechanism and operable to dispense the predetermined dose of medication or treatment fluid from a quantity of fluid confined inside the medication dispensing vessel into a patient intramuscularly or subcutaneously. The medication dispensing vessel and needle-insertion mechanism may be coupled to a microfluidic tubing or channel tract via a watertight seal using sealant, adhesive, integrated adaptor components, or other techniques for watertight joining.

A needle-insertion mechanism can comprise of springs, which when compressed upon assembly of the device, provides the necessary force needed to drive a needle in the dispensing direction upon actuation. A needle-insertion mechanism may also comprise of a proximal seal to preserve sterility of the needle.

In some situations, a medication dispensing vessel accepts gas, biasing springs, or other activations mechanisms, and comprises of a freely moving barrier and an endcap, which can be coupled to the microfluidic tubing or channel tract, and the medication or treatment fluid can be dispensed through a microfluidic tubing or channels and a needle via a barrier moved by compressed gas, spring bias, or other activation mechanisms after a seal is punctured.

An injection mechanism may further comprise of one or more winged latches that can be activated upon removal of the safety locking strip, which when activated displaces a component supporting a compressed trigger holding a needle-insertion mechanism in tension by maintaining compression of springs.

In some situations, a device includes a needle cover that is deployed after usage to prevent reuse or accidental needle sticks, wherein the safety locking strip can be used as a needle cover, or a section of a device body can be slid over a needle.

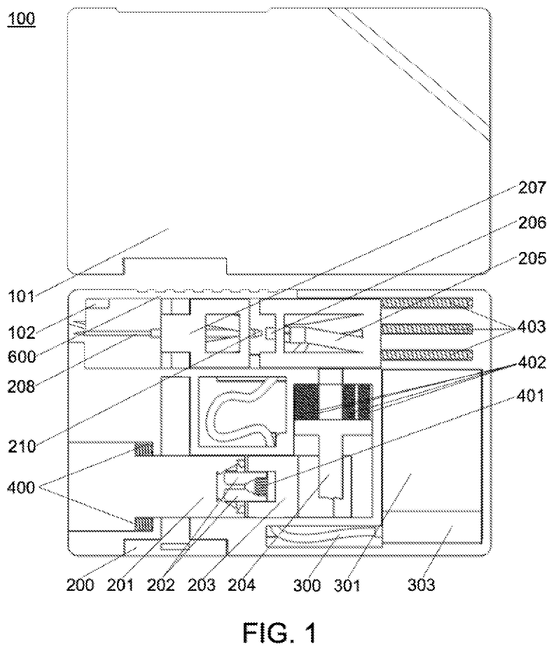

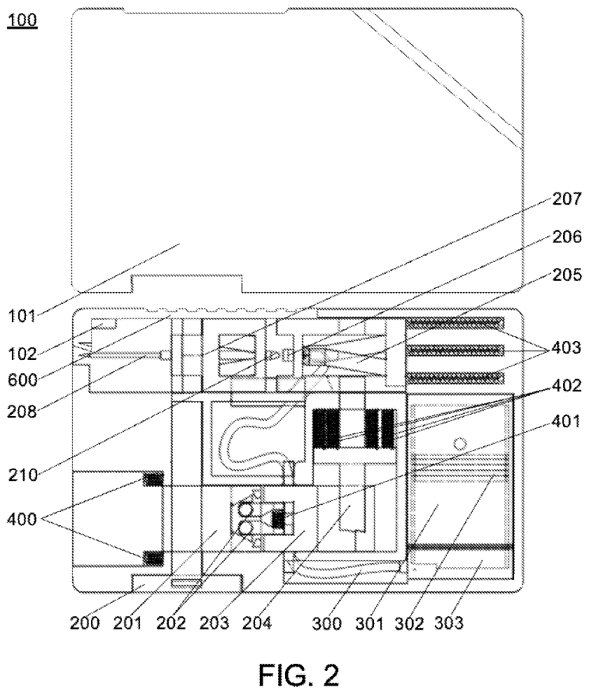

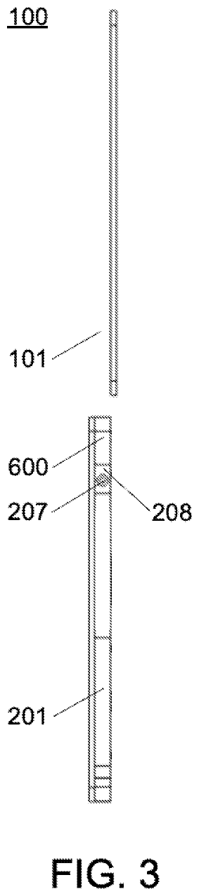

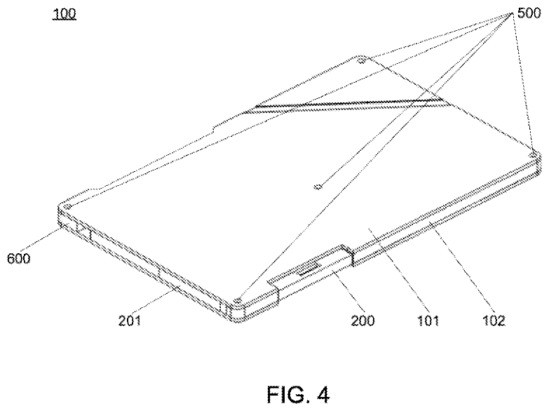

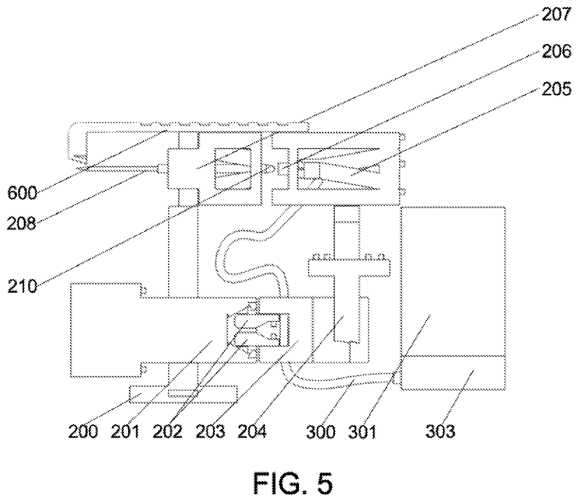

Referring, for example, to FIG. 1, FIG. 2, and FIG. 5, examples of autoinjectors, generally indicated at 100, partially in cross-section, with certain components intermittently omitted for clarity. Autoinjector 100 is not illustrated to scale, and certain elements are depicted in positions that are merely convenient for illustration and explanation and do not necessarily illustrate operational positions. The figures show a structure that is generally symmetric about a centerline, but symmetry is not required.

The inventions can be compliant with ISO/IEC 7810/ID-1/CD80 international credit card size standardizations of 85.60 mm length by 53.98 mm width, with a thickness of less than or equal to 3.80 mm (5 credit cards thick).

In some examples of the inventions, the basic structure of autoinjector 100 includes a lid 101, body 102, and fasteners 500. A constituent mechanisms within an autoinjector 100 can be structured for reciprocation within an autoinjector lid 101 and body 102. A release mechanism 201 can be kept in a stowed position by a safety strip 200. A release mechanism 201 can house wings 202 kept in a stowed position within secondary release mechanism 203. In its stowed position, a secondary release mechanism 203 helps to prevent a main trigger 204 from actuating which further prevents a distal needle-insertion mechanism component 205 from firing and initiating needle insertion. A proximal needle-insertion mechanism component 207 can be locked in place by safety strip 200, and upon mechanism activation can be compressed by distal component 205, driving needle 208 into patient and simultaneously puncturing a seal 206 to initiate medication flow.

A quantity of therapeutic fluid or medication 301 can be carried inside body 303, and can be ejected in conventional manner through tubing 300 or channel tract and needle 208 under influence of freely moving barrier 302 due to compressed gas, spring bias, or other activation mechanisms after seal 206 is punctured. A thin seal can be included proximally to preserve sterility of a needle 208 prior to use of an autoinjector 100.

In some examples, release mechanism springs 400, release mechanism wings springs 401, trigger mechanism springs 402, and needle-insertion mechanism springs 403 contain small standoff pegs to enable interface with biasing springs. The springs biasing a release mechanism 201, release mechanism wings 202, trigger mechanism 204, and needle-insertion mechanism 205 can be compression springs. Alternative biasing arrangements can also be used. Raised channels contiguous with the body 102 may interface with distal component 205 in order to provide springs 403 with lateral support during compression and extension.

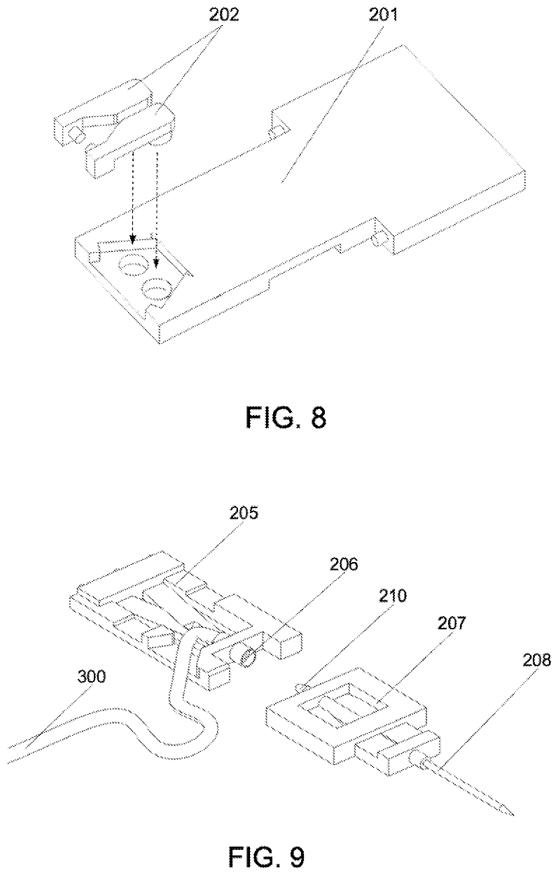

Referring, for example, to FIG. 8, a release mechanism 201 houses and enables free movement of wings 202 upon safety strip 200 removal and release mechanism arming. An inferior channel of release mechanism 201 interfaces with and overlays a safety strip 200 to enable prevention of arming in the stowed position (see FIG. 1).

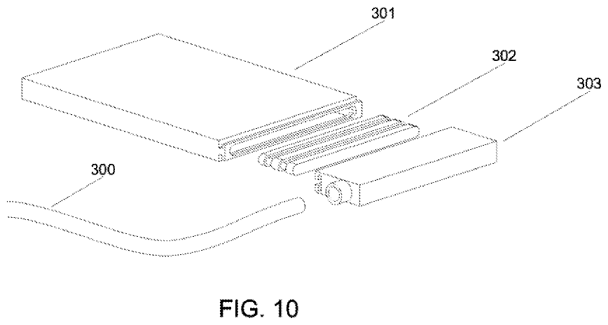

Referring, for example, to FIG. 9, microfluidic tubing 300 integrates with a distal needle-insertion mechanism component 205, in a watertight or substantially watertight manner culminating in seal 206, comprising a puncturable face and an o-ring with a housing to facilitate sealing between distal component 206 and proximal component 207 to initiate medication delivery through needle 208. Watertight joining of microfluidic tubing 300 or to distal component 205 can be accomplished through use of sealants, adhesives, integrated adaptor components, or other means of watertight joining. Inferior cutouts of distal component 205 enable unhindered movement of microfluidic tubing 300 along with a component as a needle-insertion mechanism is actuated, preventing occlusions within the tubing. Alternatively, microfluidic channels can be incorporated into body 102 as a channel to provide a medication delivery path from a medication dispensing vessel 303 to a distal component 206.

Referring, for example, to FIG. 10, a medication dispensing vessel 303 can be assembled in two parts with a medication housing 301 further accepting gas, biasing springs, or other activation mechanisms, a freely moving barrier 302, a medication or treatment fluid, and later sealed with endcap 303. The two components can be interfaced with extended joints, or be joined through the use of sealants, adhesives, welding, soldering, or other techniques. Microfluidic tubing 300 or channel can be interfaced with a medication dispensing vessel endcap 303 in a watertight seal using sealant or adhesive. Assembly of an injector can include adding housing 301, gas, biasing springs, or other activation mechanism, moveable barrier 302, medication or treatment fluid, endcap 303, and tubing 300 or channel.

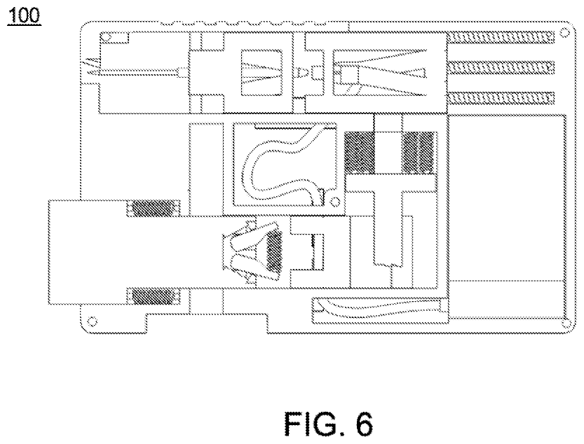

In one exemplary use of an autoinjector 100, a user can grasp an autoinjector 100 and remove a safety strip 200 from an autoinjector body 102. This serves to arm an autoinjector 100 as seen in FIG. 6, both unlocking a proximal needle-insertion mechanism component 207 and enabling a release mechanism 201 to extend out beyond a proximal edge of an autoinjector body 102 due to the biasing force of springs 400.

In the same action, release mechanism wings 202 can be withdrawn from secondary release mechanism 203, and be spread apart due to the biasing force of spring 401. In this armed state, an autoinjector 100 can be grasped by a user, oriented so a proximal face of an autoinjector faces a patient's body, and swung briskly downward towards an intended injection site. As a body makes contact with a proximal edge of autoinjector 100, an armed release mechanism 201 is depressed, enabling spread wings 202 to make contact with and displace a secondary release mechanism 203. This action causes secondary release mechanism 203 to move distally, thereby sliding a catching interface of trigger mechanism 204 out of the way, enabling a trigger mechanism 204 to actuate due to the biasing force of springs 402.

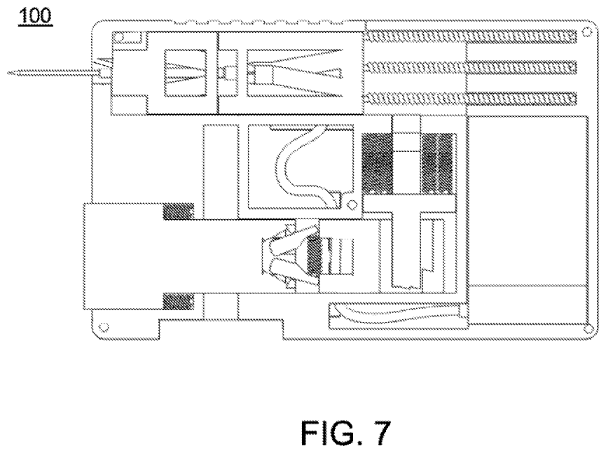

As a trigger mechanism 204 actuates, this action frees a distal needle-insertion mechanism component 205 to move under a biasing force of springs 403. As a distal needle-insertion mechanism component 205 moves proximally, it pushes a seal 206 into contact with a sharp distal cone 210 of a proximal needle-insertion mechanism component 207, thereby puncturing seal 206, compressing distal component 205 and proximal component 207 together to form a tight seal about seal 206, and driving an entire assembly forward out a proximal opening of autoinjector 100 as seen in FIG. 7. This action drives needle 208 into a patient at an intended site of injection, and, due to the puncturing of a seal 206, opens a closed system that previously existed from a medication dispensing vessel 303 through a seal 206, enabling compressed gas, biasing springs, or other activation mechanisms within a vessel to act on barrier 302 and force medication out of housing 303 through tubing 300 or channel tract and needle 208 into a patient. A user then holds an autoinjector 100 in its activated and currently dispensing state at an intended site of injection for a brief period of time until a medication or treatment fluid has been fully dispensed.

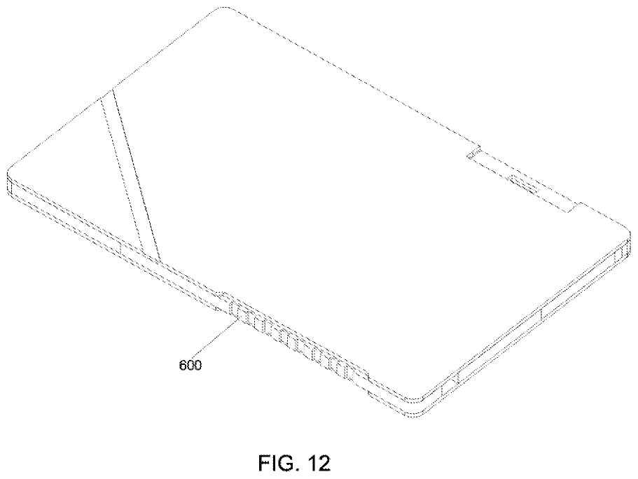

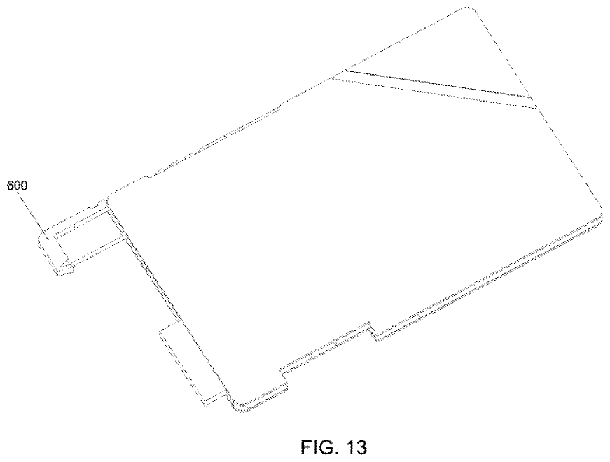

Exact duration of medication or treatment fluid delivery can depend on a necessitated volume of dosage and dispensing rates of specific embodiments. After a required time for full medication or treatment fluid delivery, a patient can grasp the autoinjector 100, pull a proximal end off of a patient's body to remove a needle 208, and, referring to FIG. 12 and FIG. 13, slide forward an outer proximal edge 600 of a body with a compliant levered endcap to cover a needle and prevent re-use or accidental needle sticks. Autoinjector 100 should be disposed of properly at a safe location after use.

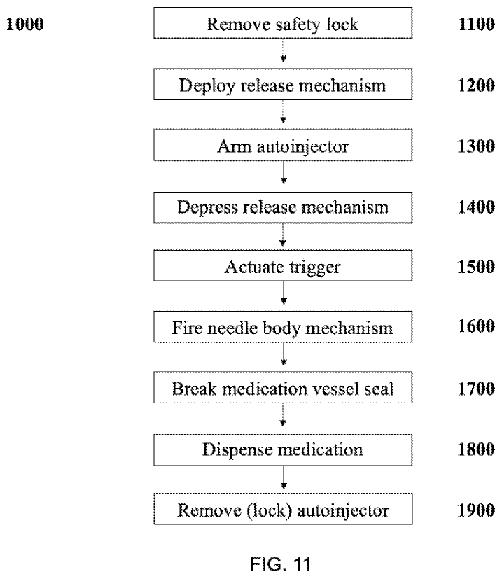

Referring, for example, to FIG. 11, the operation of a device may include beginning by removing the device from any commercially available wallet or credit card holder, not envisioned to be limiting. No external case or housing is required to be removed and no custom carrying case or device is required for the operation or the device. The safety locking mechanism 1100 is then removed, which enables a release mechanism to be deployed 1200 to subsequently arm said device 1300. Once the device is oriented, depression of an armed release mechanism 1400 actuates a trigger 1500 which fires a needle body 1600 and simultaneously punctures a medication housing seal 1700. Medication is then dispensed 1800 into a patient and after a set amount of time the device is removed 1900. A section of an outer body can then be slid forward to protect an exposed needle, or alternatively, a safety locking mechanism can be used as a sheath to cover a needle.

The suggested arrangement of components within the device body is not meant to be limiting. For example, further embodiments may include central or symmetric arrangements; a proximal trigger component centered vertically within the device body with a lateral trigger mechanism actuating a distal central mainspring, further interfacing to a centered medication vessel joined to proximal component approximating a sharp distal cone 210 or distal needle-insertion mechanism component 205. Upon device activation, a coordinated action of a centered mainspring or other biasing mechanism could advance the proximal components and medication vessel forward, puncture a seal, and deliver a centered needle through the proximal trigger mechanism into the patient to administer medication fluid. Many embodiments using various arrangements of the described components may fit within the device body and accomplish the primary goal of the device.

In the foregoing description, numerous examples and details are set forth to provide a clear understanding of various aspects of various inventions together with a written description of the claimed subject matter and to enable a person of ordinary skill in this field to make and use the same. It will also be understood, by those skilled in the relevant arts, that the present inventions may be practiced without, or with various alternatives, modifications, and/or equivalents of various of these details. In other instances, structures and devices are omitted or shown or discussed more generally in order to avoid obscuring or unduly limiting the inventions. In many cases, a description of operations is sufficient to enable one to implement the various forms of the inventions. It should be noted that there are many different, alternative, or equivalent configurations, devices, and technologies to which the disclosed inventions may be applied. The full scope of the inventions is not limited to the examples that are described herein.

I claim:

1. A device for administering medication to a patient, the device comprising:
a device body having approximately the dimensions of a credit card, wherein the dimensions include a thickness of less than or equal to 3.8 mm;
an injection mechanism within the device body, wherein the injection mechanism includes:
(i) the trigger mechanism that is operable to actuate the device; and
(ii) a needle-insertion mechanism that is operable to inject a needle into the patient intramuscularly or subcutaneously;
a safety locking mechanism within the device body that is employed to prevent accidental actuation of the needle, wherein the safety locking mechanism includes a safety locking strip positionable from a first armed position to a second disarmed position;
a generally rectangular medication dispensing vessel; and
a trigger mechanism, wherein, once the safety locking mechanism is set up, the trigger mechanism is operable through contact with the patient's body to actuate the device and dispense a predetermined dose of medication or treatment fluid from the medication dispensing vessel to the patient.

2. The device of claim 1, further comprising dimensions of a length of approximately 85.60 mm, and a width of approximately 53.98 mm.

3. The device of claim 1, wherein the needle-insertion mechanism further comprises springs, which when compressed upon assembly of the device, provides the necessary force needed to drive the needle in a dispensing direction upon actuation; and wherein the trigger mechanism further comprises a catching interface mechanism.

4. The device of claim 3, wherein the injection mechanism further comprises one or more winged latches that are activated upon removal of the safety locking strip, which when activated displaces a component supporting a compressed trigger holding the needle-insertion mechanism in tension by maintaining compression of the springs.

5. The device of claim 1, wherein the medication dispensing vessel is further associated with the needle-insertion mechanism and operable to dispense the predetermined dose of medication or treatment fluid from a quantity of fluid confined inside the medication dispensing vessel.

6. The device of claim 1, wherein the needle-insertion mechanism further comprises a proximal seal to preserve sterility of the needle.

7. The device of claim 6, wherein the medication dispensing vessel further comprises:
(i) a medication housing accepting a gas, biasing springs, or other activation mechanism;
(ii) a freely moving barrier; and
(iii) an endcap coupled to the microfluidic tubing or channel tract;
wherein the medication or treatment fluid is dispensed through the microfluidic tubing or channels and needle via a barrier moved by compressed gas, spring bias, or other activation mechanisms after the seal is punctured.

8. The device of claim 7, wherein the medication dispensing vessel further comprises:
(i) a medication housing accepting a gas, biasing springs, or other activation mechanism;
(ii) a freely moving barrier; and
(iii) an endcap coupled to the microfluidic tubing or channel tract;
wherein the medication or treatment fluid is dispensed through the microfluidic tubing or channels and needle via a barrier moved by compressed gas, spring bias, or other activation mechanisms after the seal is punctured.

9. The device of claim 1, further comprising a microfluidic tubing or channel tract coupled to the medication dispensing vessel and the needle-insertion mechanism via a watertight seal using sealant, adhesive, integrated adaptor components, or other techniques for watertight joining.

10. The device of claim 1 further comprising a needle cover that is deployed after usage to prevent reuse or accidental needle sticks.

11. The needle cover of claim 10, wherein the needle cover comprises the safety locking strip.

12. The needle cover of claim 10, wherein the needle cover comprises a section of the device body slid over the needle.
